(19) United States
(12) Patent Application Publication
(10) Pub. No.: US 2004/0019388 A1
(43) Pub. Date: Jan. 29, 2004

(54) Methods and implants for retarding stomach emptying to treat eating disorders

(76) Inventor: Warren L. Starkebaum, Plymouth, MN (US)

Correspondence Address: Thomas F. Woods, Medtronic, Inc., MS: LC340, 710 Medtronic Parkway, Minneapolis, MN, 55432-5604, US

(21) Appl. No.: 10/202,316

(22) Filed: Jul. 24, 2002

(51) Int. Cl. A61F 2/04

(52) U.S. Cl. 623/23.65; 623/11.11

(57) ABSTRACT

Methods and implants for treating patients suffering from eating disorders, particularly obesity, by constricting the size of the pylorus lumen through implantation of bulking agents or devices within the submucosa alongside the muscle layers of the pyloric sphincter to slow stomach emptying or elsewhere in the digestive tract to prolong feelings of satiety and reduce feelings of hunger.

### FIELD OF THE INVENTION

The present invention pertains to methods and implants for treating patients suffering from eating disorders, particularly obesity, by constricting the size of the pylorus lumen through microsurgical implantation of bulking agents or devices within the submucosa alongside the muscle layers of the pyloric sphincter to slow stomach emptying or elsewhere in the digestive tract to prolong feelings of satiety and reduce feelings of hunger

### BACKGROUND OF THE INVENTION

Obesity among adults and children is an increasing problem due generally to increases in caloric intake coupled with declines in exercise levels. Morbid obesity among the same population is also increasing as these habitual tendencies are coupled with physiologic conditions of certain individuals predisposed to obesity that may not fully understood in a given case. The primary treatment has always involved behavioral change involving dietary restraints to reduce caloric intake coupled with aerobic and anaerobic exercise routines or physical therapy regimens to increase caloric expenditure, resulting in a net caloric reduction. Diet and exercise plans fail since most individuals do not have the discipline to adhere to such rigorous discipline. Consequently, the marketplace is flooded with resurrected or new dietary supplements and ethical (or prescription) and patent (or nonprescription) drugs or other ingestible preparations promoted as capable of suppressing appetite or inducing satiety (i.e., the satisfied feeling of being full after eating) or of “burning” fat.

In general, these techniques for treating compulsive overeating/obesity have tended to produce only a temporary effect. The individual usually becomes discouraged and/or depressed in the course of the less radical therapies primarily focused on behavioral change after the initial rate of weight loss plateaus and further weight loss becomes harder to achieve. The individual then typically reverts to the previous behavior of compulsive overeating and/or indolence.

The gastrointestinal tract, also called the alimentary canal, is a long tube through which food is taken into the body and digested. The alimentary canal begins at the mouth, and includes the pharynx, esophagus, stomach, small and large intestines, and rectum. In human beings, this passage is about 30 feet (9 meters) long.

Small, ring-like muscles, called sphincters, surround portions of the alimentary canal. In a healthy person, these muscles contract or tighten in a coordinated fashion during eating and the ensuing digestive process, to temporarily close off one region of the alimentary canal from another region.

For example, a muscular ring called the lower esophageal sphincter surrounds the opening between the esophagus and the stomach. The lower esophageal sphincter (or LES) is a ring of increased thickness in the circular, smooth muscle layer of the esophagus. Normally, the lower esophageal sphincter maintains a high-pressure zone between 15-30 mm Hg above intragastric pressures inside the stomach.

The pylorus shown in FIGS. 1 and 3 is a specialized region at the junction of the antrum and the duodenal bulb that serves the physiologic role of a sieve to regulate the passage of chyme from the stomach. The pylorus possesses unique neural and smooth muscle characteristics as well as a distinct shape that distinguishes it from the antrum and the duodenum. A pyloric sphincter surrounds the pylorus lumen into the duodenum and is formed of proximal and distal smooth muscle loops joined by a muscular torus on the lesser curvature. The characteristics and function of the pylorus are described in the Textbook of Gastroenterology, Volume 1, T. Yamada ed., Lippincott, 1995, pp. 188-191, in “Sensory Nerves of the Intestines: Role in Control of pyloric Region of Dogs” by G. Tougas et al. (Sensory Nerves and Neuropeptides in Gastroenterology, M. Costa et al. ed. Plenum Press New York, 1991, pp. 199-211), and in “Neuromuscular Differentiation of the Human Pylorus” by K. Schulze-Delrieu et al. (GASTROENTEROLOGY 1983:84, pp. 287-92). K. Schulze-Delrieu et al refer to the proximal smooth muscle loop and the distal smooth muscle loop as the “intermediate sphincter” and “distal sphincter” respectively.

When a person swallows food, muscles of the pharynx push the food into the esophagus. The muscles in the esophagus walls respond with a wavelike contraction called peristalsis. The lower esophageal sphincter relaxes before the esophagus contracts, and allows food to pass through to the stomach. After food passes into the stomach, the lower esophageal sphincter constricts to prevent the contents from regurgitating into the esophagus.

Food is ingested until a feeling of satiety is induced and/or the stomach is distended. During ingestion and for a time thereafter, the smooth muscle layers of the pyloric sphincter are contracted to restrict the pylorus lumen and keep food in the stomach until it is liquefied. The ingested food bolus is propelled aborally mixed and ground in the antrum against the closed pylorus, and then retropropelled orally into the more proximal corpus. The muscles of the stomach rhythmically churn ingested food and digestive juices into a semi-liquid mass called chyme. The stomach muscles contract peristaltic waves triggered by a gastric pacemaker region shown in FIG. 1 and move downward or reterograde toward the pylorus and mix and shear the food into chyme while the pylorus lumen is closed. After the ingested food is ground into chyme, the pyloric sphincter relaxes in concert with antral motor activity of each peristaltic wave and lets some chyme pass into the duodenum. The pylorus lumen is small enough to function as a sieve to only let minute food particles enter the duodenum in the absence of active contraction of the pyloric sphincter.

FIG. 1 also illustrates electrogastrogram (EGG) signals that cause the depicted peristaltic wave contraction of the stomach wall. Such EGG signals normally originate in the putative pacemaker region near the junction along the greater curvature of the proximal one third or fundus and the distal two thirds of the stomach comprising the corpus and antrum. The EGG signals include slow waves that normally appear every 10-30 seconds or at a frequency of 2-6 cycles per minute (cpm), typically about 3 cpm, and propagate along the stomach wall in a characteristic pattern down to the corpus and pyloric antrum. The slow waves cause the stomach wall to rhythmically contract and move food remaining in the stomach toward the pylorus and duodenum in the peristaltic wave depicted in FIG. 1. The peristaltic wave contraction functions to create shear on the stomach contents and thus break the contents down into smaller particles that can pass through the pylorus lumen.

For example, 3 cpm slow waves are illustrated in FIG. 1 that can be sensed at three locations B, C, D but are not sensed at location A as long as the stomach is functioning normally. The three sensed EGG signals at locations B, C, D exhibit normal timed synchronization. During a peristaltic contraction, the slow waves further feature a higher voltage, high frequency action or spike potential.

Each slow wave shown in FIG. 1 at B, C and D features a corresponding high frequency action potential shortly thereafter. The slow waves, as discussed above, typically have a frequency of 3 cpm. The higher frequency action potentials, however, typically have a frequency of between 100-300 Hz.

The peristaltic wave contractions are not conducted through the pylorus to the duodenum. The duodenum rhythmically contracts in a similar fashion under the control of a separate duodenal pacemaker and a rate of about 12 cpm. The relaxation of the pyloric sphincter is independent of the duodenal contractions and is independent of but timed to peristaltic contractions of the antrum.

In advanced or extreme cases, treatment of obesity has included wiring the jaws shut for a time. Liposuction (suction lipectomy) procedures are also sometimes employed to remove adipose tissue from obese patients. Liposuction also enjoys wide application for cosmetic reshaping of the anatomy, particularly the abdomen, hips, thighs and buttocks of non-obese persons. Patients undergoing liposuction and jaw wiring may enjoy their lower weight and bulk for a time, but eventually typically regain the excised or lost weight and volume.

More radical surgical approaches are also commonly performed alone or sometimes in combination to restrict food intake or to limit absorption of nutrients in morbidly obese patients. Surgical approaches to restrict food intake include gastric banding, gastric bypass, and vertical-banded gastroplasty to decrease the size of the stomach to reduce the amount of food the stomach can hold and/or to delay the emptying of the stomach. Surgical approaches to limit nutrient absorption typically connect the stomach to the lower part of the small intestine thereby bypassing the duodenum and part of the small intestine.

Although these surgical approaches work well for some patients, many patients experience serious unpleasant side effects that, together with the risk, recuperation pain, and expense of such major surgery, discourage their widespread adoption. Risks attendant to restricting food intake include failure or weakening of the staple or suture lines causing leakage of stomach contents into the abdomen or pouch stretching.

U.S. Pat. No. 5,820,584 discloses implantation of a tubular duodenal insert extending through the pyloric valve and the duodenum to hasten passage of food from the stomach and through the duodenum before the food is fully mixed and sheered into chyme. The duodenal insert consists of an open-ended tube having a pair of spaced apart rings disposed at one of the ends of the tube. The level of intermixing of digestive fluids with partially digested food materials is controlled by one or more bores optionally disposed through the wall of the tube. Additionally, slits are optionally provided at the opposite end of the tube from the rings to permit additional intermixing. The duodenal insert can be inserted via the mouth through the esophagus and the stomach, and positioned within the duodenum. The rings are manipulated such that the rings are separately disposed on each side of the pyloric opening to anchor the duodenal insert. The duodenal insert may be removed from the body by retracting the device in reverse order through the stomach, esophagus and mouth. In some instances, a practitioner can implant the duodenal insert surgically, which enables the duodenal insert to be manufactured from a more rigid material.

The tubular duodenal insert and conventional surgical bypass procedures carry the risk of creating nutritional imbalances because, for example, Fe and Ca are absorbed mostly in the duodenum. Bypass procedures can cause “dumping syndrome” in which stomach contents move too rapidly through the remaining small intestine causing nausea, vomiting, or diarrhea. Patients may be required to use special foods or supplements and medications to manage these complications. The need to treat morbidly obese patients is so great that about 50,000 such procedures costing in excess of one billion dollars are done each year in the United States despite these risks and complications,

It has been hypothesized that retaining food in the stomach for a prolonged time promotes a prolonged “full” feeling and discourage further food intake. It was observed that the normal peristaltic rhythm of the EGG could be intentionally disrupted by electrical stimulation applied in the antrum resulting in inhibition or slowing of stomach emptying in animal studies published by S. K. Sarna et al., in “Gastric Pacemakers”, Gastroenterology 70:226-31, 1976. Distal antral stimulation in dogs produced a delay in emptying of fluids and solids. Proximal stimulation was found to have no effect on antral emptying. K. A. Kelly et al. confirmed these findings in their article “Duodenal-gastric reflux and slowed gastric emptying by electrical pacing of the canine duodenal pacesetter potential” Gastroenterology, 72:429-33, 1977. Kelly et al. demonstrated retrograde propulsion of duodenal contents with distal duodenal stimulation and entrainment of the duodenal pacesetter potential.

It has therefore been proposed to treat obesity by interrupting the peristaltic rhythm of the EGG so as to inhibit or slow stomach emptying and prolong a feeling of satiety as described, for example, in U.S. Pat. Nos. 5,423,872 and 5,690,691. The systems disclosed in these patents contemplate implanting gastric pacemakers with one or more stimulation electrodes located so as to stimulate the stomach in a retrograde or reverse phase regime, whereby the induced mechanical contraction of the stomach works against the normal rhythmic stomach contraction caused by the propagation of the slow waves and the higher frequency action potentials depicted in FIG. 1.

It is also believed that a satiety center in the brain develops the sensation of satiety in a complicated manner believed in part to be due to increased firing of afferent vagal fibers of the vagal nerves extending between the stomach and brain when the stomach is filled. Thus, it has been proposed to electrically stimulate the stomach or the vagus nerves, as set forth in U.S. Pat. Nos. 5,263,480, 5,540,730, and 5,188,104, at a rate mimicking the observed increase to mediate afferent information from the stomach to the satiety centers.

Unfortunately, it is not a simple procedure to implant the stomach wall or vagal nerve electrodes, or to do so in an effective place to accomplish the goal of inducing the satiety sensation when the stomach is not actually full. And, the vagal nerves are involved in the regulation of the function of many body organs, including the heart, and stimulation of vagal nerves for any given purpose can have unintended consequences. Moreover, it has been reported that stimulation of the vagal nerves can increase transpyloric flow in pigs in “Vagal Control of Pyloric Resistance”, by C. H. Malbert et al. (Am. J. Physio. 269 (Gastrtointest Liver Physiol 32): G558-569, 1995).

Thus, despite these improvements, there remains a need for treating obesity that is simple to implement and overcomes the disadvantages of the above procedures.

### SUMMARY OF THE INVENTION

The methods and apparatus of the present invention overcomes these disadvantages of the prior art through the creation of a restriction restricting the passage of ingested food through the GI tract to thereby retard stomach emptying and induce a feeling of satiety or fullness that induces the obese person to slow or halt eating.

In a first aspect of the present invention, a pyloric restriction that reduces the amount of chyme that is passed from the stomach into the duodenum during normal peristaltic activity is surgically created, preferably employing minimally invasive surgical techniques. The pyloric restriction causes the stomach to empty more slowly when eating and induce feelings of satiety or discomfort occur with less food ingested than would be the case without the obstruction.

The bulking agent or device implanted into or adjacent to pyloric sphincters surrounding the pylorus lumen restricts the maximal opening of the pylorus lumen to slow or retard stomach emptying following eating to induce a feeling of satiety or to otherwise retain stomach contents or chyme in the stomach for prolonged time periods to thereby limit the patient's desire to eat and to bring about weight loss.

In a second aspect of the invention, a restriction in the small intestine that restricts the passage of contents of the intestine is surgically created, preferably employing minimally invasive surgical techniques. The restriction causes feelings of discomfort with less food ingested than would be the case without the obstruction.

In accordance with the invention, minimally invasive surgical techniques are preferably employed to implant one or more mass of bulking agent or one or more bulking device into the submucosal region of the GI tract wall where the restriction is intended to be created to slow emptying and treat obesity.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, wherein:

FIG. 1 depicts an example of the peristaltic wave created as GI tract signals, particularly the slow wave and the spike potentials characteristic of peristalsis that can be detected through electrodes coupled to the stomach wall, traverse the stomach wall;

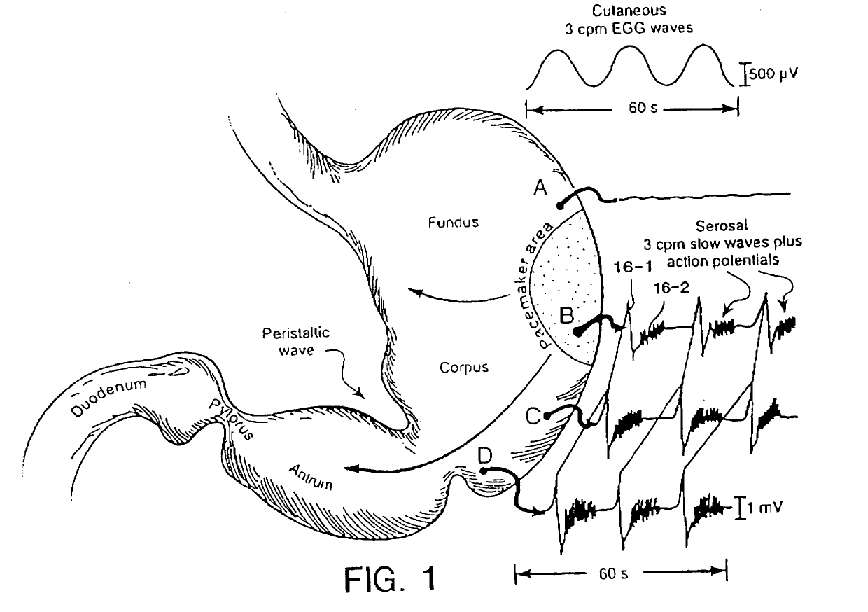

FIG. 2 is a schematic illustration of accessing the pylorus or a region of the small intestine to implant bulking agents or devices sub-mucosally adjacent to the pyloric sphincters or intestinal wall;

FIG. 3 depicts the pylorus in longitudinal and mucosal section views and showing where bulking agents or devices can be implanted sub-mucosally in relation to the labeled parts of the pylorus;

FIG. 4 is an expanded partial cross-section view of the stomach and pylorus depicting the access to the submucosal implantation sites;

FIG. 5 is an expanded cross-section view of the pylorus depicting implanted masses of bulking agent or bulking devices narrowing the esophageal lumen; and

FIG. 6 is a schematic illustration of the GI tract identifying further potential implantation sites of masses of bulking agent or bulking devices in accordance with the present invention.

### DETAILED DESCRIPTION OF THE PREFERRED EMBODIMENTS

In the following detailed description, references are made to illustrative embodiments for carrying out various aspects of the invention.

Pyloric obstructions occur in some infants and occasionally in adults wherein ingested food cannot pass through the pylorus lumen in sufficient quantity to provide adequate nutrition. The stomach fills and its contents are then regurgitated. Infants suffer malnutrition and failure to thrive unless surgical procedures are undertaken to correct the obstruction. Thus, the present invention is expected to employed in treating obese adults so that the induced partial pyloric obstruction or small intestine obstruction prolongs emptying of the stomach or small intestine to induce the patient to refrain from eating frequently or eating too much.

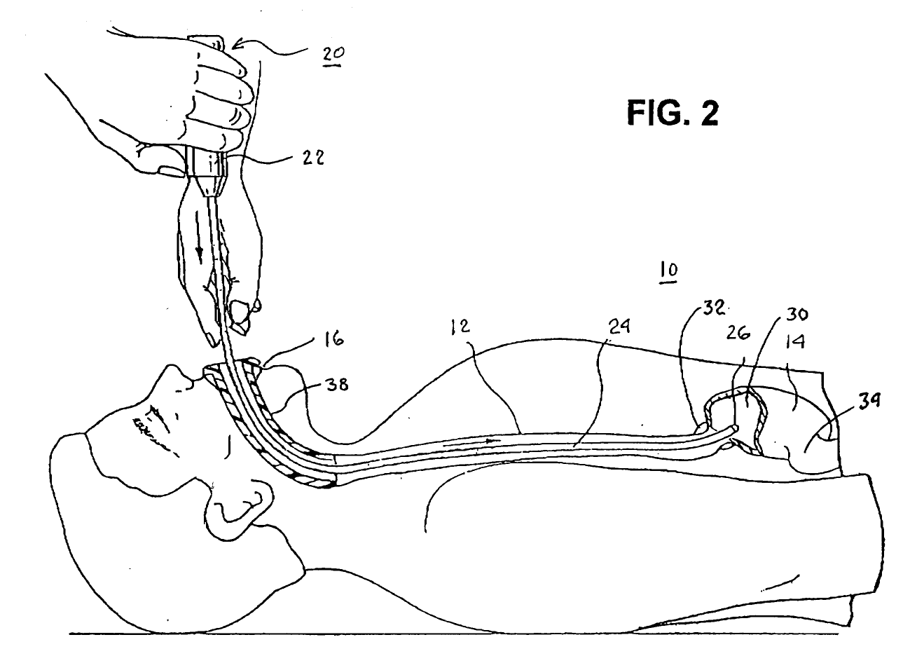

FIG. 2 is a schematic view of obtaining access into the stomach 14 of a patient 10 employing a delivery instrument 20 to enable the implantation of a preformed implant or a mass of bulking agent within the wall of the pylorus or the small intestine as described further below. The delivery instrument 20 comprises a handle 22 coupled to the proximal end of an elongated instrument body 24 extending to an instrument body distal end 26 and enclosing at least one delivery lumen. The delivery instrument 20 encloses at least one instrument lumen distal end opening at instrument body distal end 26.

The delivery instrument 20 can take the form of the instruments described in U.S. Pat. Nos. 6,251,063, 6,251,064, and 6,358,197 that are employed to inject a mass or masses of bulking agents within the wall of the esophagus in the region of the lower esophageal sphincter (LES) or into the rectal wall in the region of the anal sphincter that solidify in situ. Alternatively, the delivery instrument 20 can take the form of the instruments set forth in U.S. Pat. Nos. 6,098,629, 6,338,345, and 6,401,718 that are employed to insert pre-formed prosthetic bulking devices below the mucosa in the region of the LES. The implantation of the mass(es) of bulking agent(s) or the bulking device(s) within the mucosa in the region of the LES is intended to treat patients suffering from gastroesophageal reflux disease (GERD). The solidified mass(es) of bulking agents or bulking device(s) add bulk to the LES to elevate the LES closing pressure or function as valve mechanisms. The delivery of bulking agents through endoscopes or other instruments into periurethal tissue at the site of a defect to correct urinary incontinence or vesicoureteteral reflux is also disclosed in U.S. Pat. Nos. 5,667,778, 5,755,658, and 5,785,642. Preferably, the delivery instrument 20 incorporates the imaging features of an endoscope or gastroscope, the delivery lumen(s) for delivering the mass(es) of bulking agent(s) or bulking device(s), and a retractable cutting or penetrating tip or other mechanism that is selectively actuable to perforate the mucosa to enable advancement of the mass(es) of bulking agent(s) or bulking device(s) therethrough.

In accordance with the present invention, the instrument body 24 is inserted through a curved mouth and throat guard 38 inserted into the patient's mouth 16, and the instrument body distal end 26 is advanced through the esophagus 12 and LES 32 and into the stomach cavity 30. The instrument body distal end 26 is advanced either to the pylorus 34 or further through the duodenum and to an implantation site of the small intestine. The instrument distal end 26 is directed to the site of implantation in the intestinal wall or the wall of the pylorus 34, and the mass(es) of bulking agent or bulking device(s) are implanted in one of the ways described further below.

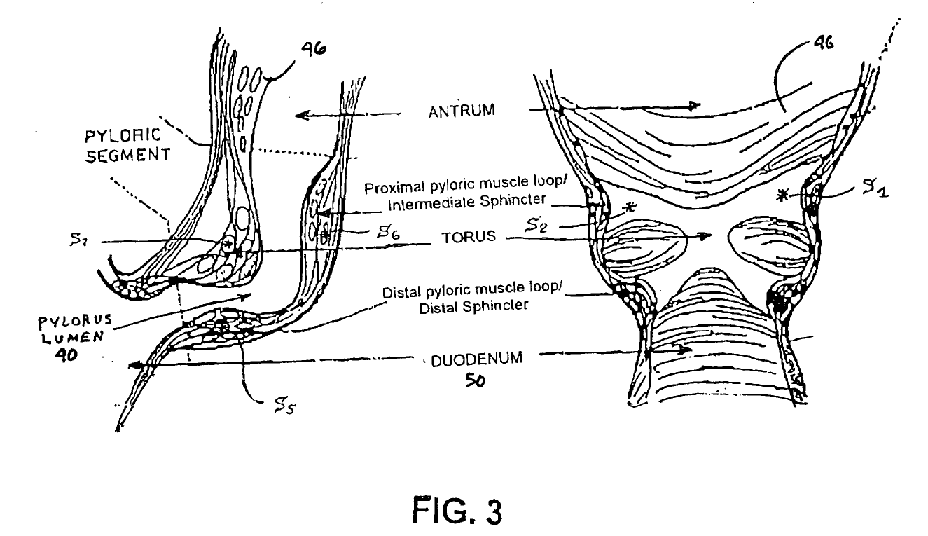

FIG. 3 depicts the pylorus 34 between the stomach 14 and the duodenum 50 in greater detail. In the illustrated embodiments, the mass(es) of bulking agent(s) or bulking device(s) can be implanted within the submucosa 44 between the mucosal surface or mucosa 46 and the pyloric sphincter 48. Within the stomach proper, the submucosa 44 is a fibrous layer of tissue separating the mucosa 46 and the muscularis externa which itself comprises oblique, circular and longitudinal muscle layers.

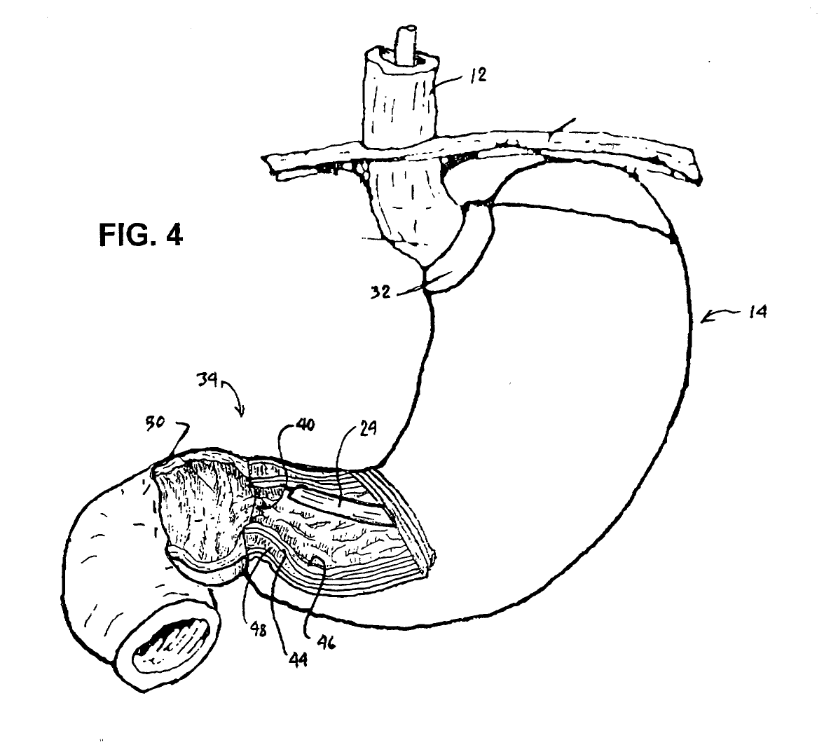

FIG. 4 depicts the pylorus 34 in longitudinal and mucosal section views reproduced from the above-referenced Tougas et al. article and showing where the mass(es) of bulking agent or bulking device(s) can be implanted in the pylorus wall 42 in relation to the labeled parts of the pylorus 34. A submucosal space, that is a potential space, can be created between the mucosa 46 and the pyloric sphincter 48 by the separation of mucosa 46 from the pyloric sphincter 48. The submucosa 44 is a springy tissue that can be separated apart by a blunt instrument or cut using mechanical cutting techniques or cautery tools in order to create a submucosal space or site for implantation of a mass of bulking agent or bulking device. It is expected that solutions of fluid bulking agents can be directly injected into the submucosa 44 to displace submucosal tissue and solidify in situ to form a mass or implant of non-biodegradable bulking agent. Alternatively, a submucosal space or site for implantation of a mass of bulking agent or bulking device can be created intramuscularly by distension and separation of muscle fibers of the pyloric sphincter 48.

The pyloric sphincter 48 comprises an intermediate sphincter loop and a distal sphincter loop joined in the shape or a torus. The mass(es) of bulking agent or bulking device(s) can be implanted adjacent the intermediate sphincter loop at sites S₁ and S₂ or in various ones of the sites S₁ through S₇ to efficaciously narrow the pylorus lumen 40. Ideally, the bulking device is implanted in a bulking device(s) can be implanted adjacent the intermediate sphincter loop at sites S₁ and S₂ or in various ones of the sites S₁ through S₇ to efficaciously narrow the pylorus lumen 40. Ideally, the bulking device is implanted in a position that extends across or is closely adjacent the pyloric sphincter 48 so that residual sphincter activity is optimized. Alternatively, the mass(es) of bulking agent or bulking device(s) can be implanted in or against the smooth muscle layers of the duodenum 50 to provide bulk cause the distal and/or intermediate sphincters to contract to obstruct the pylorus lumen 40. The precise number, shape and positioning of the mass(es) of bulking agent or bulking device(s) depends on the patient's anatomy, and will be a matter of clinical choice at the time of implantation.

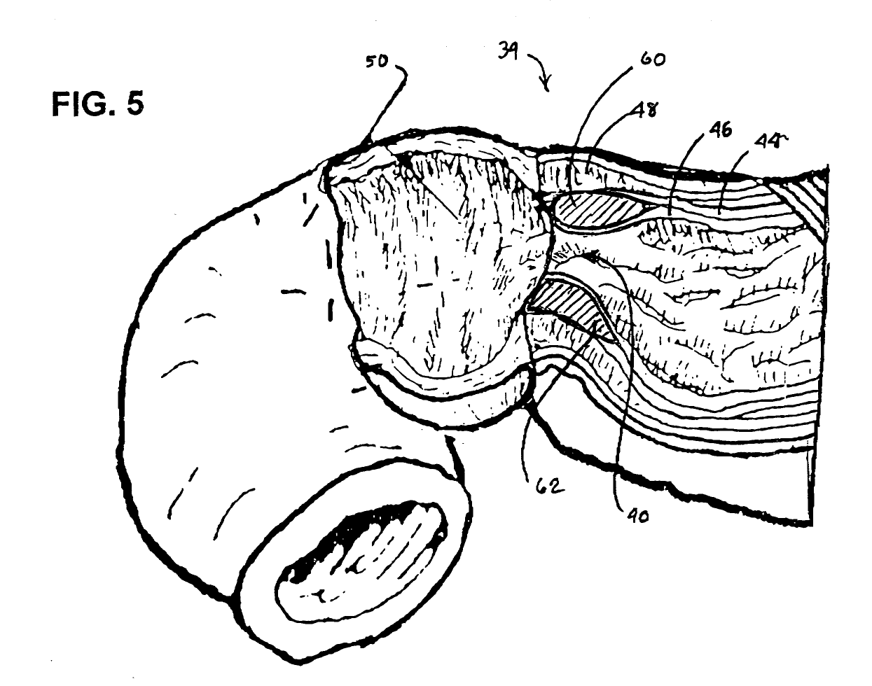

FIG. 5 depicts implanted masses of bulking agent or bulking devices 60 and 62 implanted sub-mucosally adjacent to the pyloric sphincter 48. The particular composition of the masses of bulking agent or bulking devices 60 and 62 can be selected from the following described examples or their equivalents. The particular implantation sites, and the size, shape and number of such implants can be selected by the surgeon to meet the needs of the particular patient.

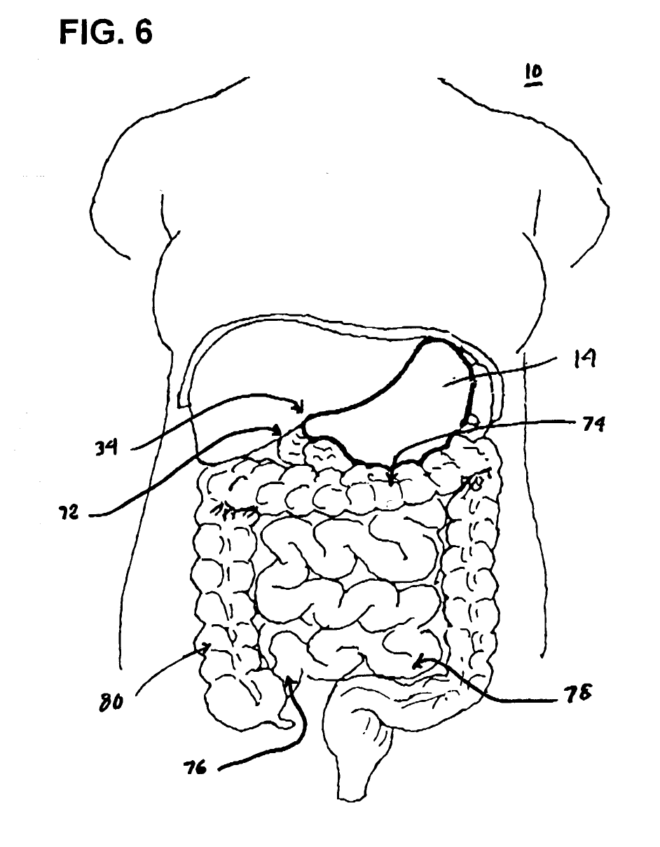

FIG. 6 is a schematic illustration of the GI tract identifying potential implantation sites of one or more mass of bulking agent or bulking device to restrict a lumen and slow emptying of the contents of the stomach 14, duodenum 50 or small intestines 78. The particular composition of the masses of bulking agent or bulking devices implanted at such sites can be selected from the following examples or their equivalents. The particular implantation sites, and the size, shape and number of such implants can be selected by the surgeon to meet the needs of the particular patient.

The implantation within the duodenum 50 can be adjacent the first flexure (flexura duodenisuperior) 72 or adjacent the duodenojunal flexure 74. One or more bulking device or mass or bulking can be implanted endoscopically within the wall of the duodenum in a manner similar to the above-described procedure for insertion of the same in relation to the pylorus 34.

One or more bulking device or mass or bulking can be implanted within the wall of the ileocecal sphincter 76 at the junction of base of the ascending colon 80 and the small intestine 78. The ileocecal sphincter 76 opens to allow partially digested chyme to move from the small intestine 78 to the colon 80. Partially constricting the ileocecal sphincter 76 when it is normally relaxed would limit the movement of partially digested food from the small to large intestine, creating a condition similar to pseudo-obstruction (with attendant symptoms of nausea, vomiting, abdominal pain in association with eating). One or more bulking device or mass or bulking can be implanted with the aid of a sigmoidscope or a laparascope within the wall of the ileocecal sphincter 76 in a manner similar to the above-described procedure for insertion of the same in relation to the pylorus 34.

Implantation of Fluid Bulking Agent:

Any suitable material can be used with the method of the present invention to form a bulking implant in situ when the fluid, separately or in conjunction with another fluid, is introduced to the implantation site. Such materials include those disclosed, for example, in the above-referenced '642, '658, '197, '063 and '064 patents or the materials disclosed in the above-referenced '778 patent. In general, the apparatus disclosed in the above-referenced '197 patent or other apparatus can be employed as the delivery instrument 20 to immobilize and perforate the mucosa, form the implantation space or site, deliver masses or boluses of one or more liquid into the site, and view these operations.

The mass forming materials can be injected directly into the submucosa to form the mass of bulking agent therein. Alternatively, a space can first be formed in the submucosa by injection of saline solution other aqueous or physiologic solution into the submucosa to form a blister. The blister of saline solution other

Preferably, inert, non-resorbable, biocompatible fluid solutions are used that when introduced into the body forms a non-biodegradable solid mass that does not flow perceptibly under moderate stress, resists compression, tension and strain forces that tend to deform it, and retains a definite size and shape under ordinary conditions but that can be compressed. The liquid solution preferably comprises at least first and second fluid compounds that are separately injected and form the non-biodegradable solid mass at the site, e.g., by precipitation.

Such a nonaqueous solution is a solution of a biocompatible polymer or prepolymer and a biocompatible solvent that can optionally include a contrast agent for facilitating visualization of the solution in the body.

Preferably, a contrast agent is incorporated into the solution that precipitates into the solid mass or otherwise solidifies at the site of delivery. Such contrast agents comprise biocompatible radiopaque materials that are either water-soluble or water insoluble. Water-soluble contrast agents include metrizamide, iopamidol, iothalamate sodium, iodomide sodium, and meglumine. Well known water insoluble contrast agents include gold, tungsten and platinum powders as well as tantalum powder, tantalum oxide, and barium sulfate, etc. The optional contrast agent in the implants permits the bulking agents to be observed entering the site of interest and to be monitored after completion of the procedure so that the stability of the mass and any changes in its shape or location can be observed over time.

The non-toxic biocompatible solvent is an organic liquid such as dimethylsulfoxide (DMSO), analogues/homologues of dimethylsulfoxide, ethanol, ethyl lactate, acetone, and the like and aqueous mixtures thereof.

Suitable biocompatible polymers are non-toxic, chemically inert, and substantially non-immunogenic when used internally in the patient and which are substantially insoluble in physiologic liquids. The particular biocompatible polymer employed is not critical and is selected relative to the viscosity of the resulting polymer solution, the solubility of the biocompatible polymer in the biocompatible solvent, and the like.

A useful bulking agent mass forming solution is a composition comprising a biocompatible polymer, a biocompatible solvent and optionally a biocompatible contrast agent. More particularly, the mass forming solution preferably comprises about 2.0 to about 9.0 weight percent of a biocompatible polymer, about 50 to about 88 weight percent of a biocompatible solvent and optionally from about 10 to about 41 weight percent of a biocompatible contrast agent having a preferred average particle size of about 5-10 μm or less.

Representative biocompatible polymers include those specifically set forth in the above-referenced '658 patent including cellulose acetates, ethylene vinyl alcohol copolymers, hydrogels, polyalkyl(C₁-C₆) acrylates, acrylate copolymers, olyacrylonitrile, polyvinylacetate, cellulose diacetate, cellulose acetate butyrate, nitrocellulose, copolymers of urethane/carbonate, copolymers of styrene/maleic acid, and mixtures thereof.

The molecular weights of such polymers can be selected from the literature and are commercially available or can be prepared by art recognized, non-proprietary procedures. Polymers having a lower molecular weight will impart a lower viscosity to the composition as compared to higher molecular weight polymers.

Accordingly, adjustment of the viscosity of the composition can be readily achieved by mere adjustment of the molecular weight of the polymer composition.

In one example, the weight average molecular weight, as determined by gel permeation chromatography, of suitable commercially available cellulose diacetate polymers having an acetyl content of from about 31 to about 40 weight percent can range between about 25,000 and about 200,000.

In another example, the weight average molecular weights of suitable polyacrylonitrile, polyvinylacetate, polyalkyl(C₁-C₆) acrylates, acrylate copolymers, polyalkyl alkacrylates wherein the alkyl and alk groups independently contain one to six carbon atoms, cellulose acetate butyrate, nitrocellulose, copolymers of urethane/carbonate, copolymers of styrene/maleic acid and mixtures thereof typically are at least about 50,000 and more preferably can range between about 75,000 and about 300,000.

Ethylene vinyl alcohol copolymers are either commercially available or can be prepared by art recognized procedures. Ethylene vinyl alcohol copolymers comprise residues of both ethylene and vinyl alcohol monomers. Small amounts (e.g., less than 5 mole percent) of additional monomers can be included in the polymer structure or grafted thereon provided such additional monomers do not alter the implanting properties of the composition. Such additional monomers include, by way of example only, maleic anhydride, styrene, propylene, acrylic acid, vinyl acetate and the like.

The overall hydrophobicity/hydrophilicity of a vinyl alcohol copolymer that, in turn, affects the relative water solubility/insolubility of the copolymer and the rate of precipitation of the copolymer in an aqueous solution is affected by the ratio of ethylene to vinyl alcohol in the copolymer. An exemplary vinyl alcohol copolymer comprises a mole percent of ethylene of from about 25 to about 60 and a mole percent of vinyl alcohol of from about 40 to about 75, more preferably a mole percent of ethylene of from about 40 to about 60, and a mole percent of vinyl alcohol of from about 40 to about 60. The ethylene vinyl alcohol copolymer composition is selected such that a solution of 8 weight-volume percent of the ethylene vinyl alcohol copolymer in DMSO has a viscosity equal to or less than 60 centipoise at 20° C. and more preferably 40 centipoise or less at 20° C.

It should be noted that the biocompatible polymer composition can be replaced with a biocompatible prepolymer composition containing a biocompatible prepolymer that polymerizes in situ alone or in the presence of a water insoluble contrast agent and a biocompatible solvent. Such a prepolymer can either be a monomer or a reactive oligomer that is non-toxic, chemically inert, substantially non-immunogenic when used internally in the patient and substantially insoluble in physiologic liquids. Cyanoacrylates, hydroxyethyl methacrylate, silicon prepolymers, and the like, are suitable biocompatible prepolymers.

The compositions employed in the methods of this invention are prepared by conventional methods known in the prior art and disclosed in the above-referenced '642, '658, '197, '063, '064 and '778 patents. The components are added together in no particular order, and the solution is stirred as necessary under an anhydrous atmosphere at ambient pressure until it is homogeneous. The resulting solution is heat sterilized and sealed in vials until injected.

Once the implant forming solution has been introduced into submucosal space, the biocompatible polymer or prepolymer of the implant forming solution precipitates to form one or more discrete mass of solid bulking agent. The amount of implant forming solution injected into the submucosal space for each implant can range from 0.01 cc to 10 cc.

Other suitable materials can be utilized for implant formation in the method of the present invention. Such materials include suitable suspensions such as injectable bioglass of the type described in Walker et al., “Injectable Bioglass as a Potential Substitute for Injectable Polytetrafluorethylene Particles”, J. Urol., 148:645-7, 1992, small particle species such as polytetrafluoroethylene (PTFE) particles in glycerine such as Polytef®, biocompatible compositions comprising discrete, polymeric and silicone rubber bodies such as described in U.S. Pat. Nos. 5,007,940, 5,158,573 and 5,116,387 and biocompatible compositions comprising carbon coated beads such as disclosed in U.S. Pat. No. 5,451,406. Such suitable materials for forming implants further include collagen and other biodegradable material of the type disclosed in U.S. Pat. No. 4,803,075 and other known injectable materials.

Still further materials that can be utilized for implant formation in the method of the present invention comprise a suspension of smooth muscle cells in a biodegradable non-proteinaceous polymer solution that forms an ionically cross linked hydrogel having the cells dispersed therein when injected in vivo, which becomes a non-migratory, volume stable tissue mass as described in the above-referenced '778 patent. Preferably the smooth muscle cells are harvested from the patient.

Preferably, the polymer is selected from the group consisting of polysaccharides, polyphosphazines, alginate, hyaluronic acid, polyacrylates, and polyethylene oxide-polypropylene glycol block copolymers and is cross linkable by temperature or pH. Suitable polymers have basic side groups that can be reacted with anions and are selected from the group of polymers consisting of poly(vinyl amines), poly(vinyl pyridine), poly(vinyl imidazole), and imino substituted polyphosphazenes. Other suitable polymers having acidic side groups that can be reacted with cations are selected from the group of polymers consisting of poly(phosphazenes), poly(acrylic acids), poly(methacrylic acids), copolymers of acrylic acid and methacrylic acid, poly(vinyl acetate), sulfonated polymers, and copolymers having acidic side groups formed by reaction of acrylic or methacrylic acid and vinyl ether monomers or polymers.

Implantable Bulking Devices

In accordance with another aspect of the present invention, one or more preformed esophageal bulking device of the type disclosed in the above-referenced '629 patent is implanted below the mucosa in the vicinity of the pyloric sphincter. The bulking device comprises a flexible, compressible body formed of a compressible filler and an outer layer. The outer layer may be provided with a porous surface structure to permit cellular ingrowth. The bulking device has a preformed shape, having blunt, atraumatic edges. In one embodiment, the filler comprises an open-celled foam, such as polyurethane.

One suitable bulking device construction comprises the use of an inflatable pillow or balloon, partially or completely filled with a liquid or semi-liquid, which allows one end to be compressed by peristaltic compression and the other end to expand bulbously. The ability of the volume of the bulking device to flow from one end of the bulking device to the other and back permits the passage of a peristaltic wave, as will be appreciated by those of skill in the art in view of the disclosure herein. Suitable elastomeric balloons can be formed from silicone, latex, or other materials known in the art.

Suitable bulking devices comprise a soft, flexible body that may have an axial length from 1.0 cm to 5.0 cm, a width (circumferential implanted direction) of 0.2 cm to 2.0 cm, and a thickness (radial implanted direction) of 1.0 mm to 8.0 mm.

Many bulking devices of the present invention have a length within the range of 1.5 cm to 4.0 cm, a width within the range of 0.4 cm to 1.5 cm, and a thickness within the range of 1.5 mm to 6.0 mm. In one embodiment, the bulking device has a length of 2.0 cm to 3.0 cm, a width of 0.8 cm to 1.0 cm, and a thickness of 4.0 mm to 6.0 mm.

Length to thickness ratios are generally no more than about 15:1 and are often no more than about 6:1 or 4:1. Length to thickness ratios on the order of less than 3:1 may also be desirable depending upon the severity of the condition. The cross-sectional area of the bulking device may also vary at different points along the length of the same bulking device. As mentioned above, optimal dimensions may be patient specific and can be determined through routine experimentation of one of skill in the art in view of the disclosure herein.

A pylorus lumen having a relaxed open diameter of 2.0 cm, for example, has a cross-sectional lumen area of 3.14 cm². A 25% bulking function could be accomplished by providing a bulking device 16 having a total cross-sectional area in the bulking zone of about 0.785 cm². The bulking area may represent the area of a bulking device having a generally oval or rectangular cross-section (e.g., 0.443 cm×1.772 cm) that is adapted to extend axially for a length of 1 to 3 cm beneath the mucosa.

The present inventors further contemplate embodiments of the bulking device that have surface textures, coatings or structures to resist migration. In general, the entire outer surface of the outer layer or filler can be coated or textured to facilitate tissue attachment such as by cellular ingrowth. The resulting attachment surface can be integral with the bulking device or can be directly or indirectly connected to the bulking device 16 so that the bulking device can be positioned and retained in the desired position within the esophageal wall. The outer surface may additionally, or alternatively, be provided with any of a variety of tissue retention structures such as hooks, barbs, tacks, clips, sutures, staples, tissue adhesives, attachment strips, attachment spots, attachment connectors, or other attachment means which will be understood by those of skill in the art in view of the disclosure herein.

The porosity of the cellular ingrowth surface may range from about 20 μm to about 100.0 μm or greater. Desirably, the porosity of the cellular ingrowth surface ranges from 20 μm to 50 μm and, in many embodiments, the porosity of the cellular ingrowth surface ranges from 20 μm to 30 μm.

Suitable outer layer and/or attachment surface materials include polytetrafluoroethylene (PTFE), polyethylene terephthalate, polyester, polyurethane, silicone, Dacron, polypropylene knit, and other materials which will be apparent to those of skill in the art in view of the present disclosure. In one embodiment of the invention, the cellular ingrowth surface comprises PTFE having a 22 μm pore size. This porosity appears to permit shallow ingrowth into the bulking device to prevent axial migration of the device along tissue planes yet allows for relatively easy explantation.

Implantation of the bulking device below the mucosa can be accomplished in any of a variety of ways, as will be apparent to those of skill in the art in view of the disclosure herein. Delivery systems can be specially constructed or assembled from existing endoscopic and other surgical tools to accomplish the basic implantation steps.

In general, the implantation site for a particular patient is identified, such as by endoscopy and manometry. Tissue adjacent to the implantation site is preferably immobilized to permit a puncture or incision to be made. Immobilization of the mucosa may be accomplished by grasping the tissue utilizing forceps, such as those that may be advanced through a working channel on an endoscope. Alternatively, a vacuum may be applied to a lumen through an endoscope to grasp the mucosa.

The mucosa is pierced to enable insertion of the prosthesis using counter-traction on the tissue applied by way of the tissue grasper. The mucosal layer may be pierced in a variety of ways, as will be recognized in the art. In accordance with one aspect of the present method, a needle is utilized to pierce the mucosa. Alternatively, an electrocautery cutter or any of a variety of sharp dissection tools may be utilized to pierce the mucosa and provide access to the submucosa.

A blister or pouch within the submucosa can then be created by injecting a volume of fluid, such as saline solution, through the pierced mucosa. Alternatively, any of a variety of blunt tools may be utilized to achieve a blunt dissection in the submucosa or between adjacent tissue planes to form a pouch for receiving the bulking device. Alternatively, an inflation device, such as a balloon, may be specially shaped for insertion and inflation to separate submucosal tissue and provide a submucosal pouch.

One or more bulking device is then introduced into the submucosal pouch by way of a grasper, clamshell deployment device, or other tools. A flexible and suitably shaped bulking device may be disposed within a catheter or instrument lumen and pushed or pulled out of the distal end lumen opening and into the submucosal pouch.

The mucosal opening is preferably closed using any of a variety of closure techniques following placement of the bulking device into the submucosal pouch. A conventional suture, ligating bands, staples or clips may be utilized endoscopically, as will be understood in the art. Alternatively, a topical glue or other adhesive patch may be utilized to close the opening in the mucosa.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments. It will also be understood that there may be other structures, functions and operations ancillary to the typical operation of the above-described devices are not disclosed and are not necessary to the practice of the present invention. In addition, it will be understood that specifically described structures, functions and operations set forth in the above-referenced patents can be practiced in conjunction with the present invention, but they are not essential to its practice.

Thus, embodiments of METHODS AND IMPLANTS FOR RETARDING STOMACH EMPTYING TO TREAT EATING DISORDERS are disclosed. One skilled in the art will appreciate that the present invention can be practiced with embodiments other than those disclosed. The disclosed embodiments are presented for purposes of illustration and not limitation, and the present invention is limited only by the claims that follow.

1. A method of treating obesity comprising providing a pyloric bulking device having a predetermined form and inserting the pyloric bulking device below the mucosa in the vicinity of a pyloric sphincter to substantially close the pylorus lumen to slow emptying of the stomach when the pyloric sphincter is relaxed.

2. The method of claim 1, wherein the inserting step comprises inserting two or more pyloric bulking devices.

3. The method of claim 1, further comprising the step of attaching the pyloric bulking device to adjacent tissue.

4. The method of claim 3, wherein the attaching step comprises permitting cellular ingrowth into a porous surface on the pyloric bulking device.

5. The method of claim 1, further comprising the step of explanting the pyloric bulking device from the vicinity of the pyloric sphincter.

6. The method of claim 1, wherein the pyloric bulking device comprises a flexible outer shell and a filling material.

7. The pyloric bulking device of claim 1, wherein the pyloric bulking device comprises a flexible outer shell filled with a filling material, the flexible outer shell having an attachment surface that allows tissue ingrowth from adjacent tissue in the vicinity of the pyloric sphincter.

8. The method of claim 1, wherein the pyloric bulking device has a shape and volume selected to partially obstruct the pylorus lumen when inserted below the mucosa in the vicinity of a pyloric sphincter.

9. A pyloric bulking device for implantation below the mucosa in the vicinity of the pyloric sphincter to substantially close the pylorus lumen to slow emptying of the stomach, comprising a flexible body.

10. The pyloric bulking device of claim 9, wherein the flexible body further comprises a filler and an attachment surface which allows tissue ingrowth from adjacent tissue in the vicinity of the pyloric sphincter.

11. The pyloric bulking device of claim 9, wherein the flexible body has a preformed elongate structure with blunt, atraumatic edges.

12. The pyloric bulking device of claim 9, comprising an oblong shape having a proximal end and a distal end such that the proximal end has a smaller cross-section than the distal end.

13. The pyloric bulking device of claim 10, wherein the attachment surface comprises a porous surface.

14. The pyloric bulking device of claim 10, wherein the filler comprises a material selected from the group consisting of silicone, polyurethane, polysulfone, hydrogels, and polyester.

15. The pyloric bulking device of claim 10, wherein the filler comprises a biocompatible foam.

16. The pyloric bulking device of claim 10, wherein the attachment surface has a pore size within the range of from about 20 μm to 100 μm.

17. The pyloric bulking device of claim 10, wherein the attachment surface and flexible body comprise a unitary structure.

18. The pyloric bulking device of claim 10, wherein the attachment surface and flexible body comprise at least two components connected together.

19. The pyloric bulking device of claim 10, wherein the filler is a biocompatible liquid or gel selected from the group consisting of saline, silicone oil, DMSO, polyvinyl, pyrollidone and hydrogels.

20. A method of decreasing the pylorus lumen of the pyloric sphincter, comprising the steps of:
trans-pyloricly introducing an endoscope to a treatment site in the vicinity of the pyloric sphincter;
providing an access pathway through the mucosa; and
introducing a pyloric bulking device into the wall of the pylorus below the mucosa, so that the pyloric bulking device cooperates with the pyloric sphincter to substantially close the pylorus lumen to slow emptying of the stomach when the pyloric sphincter is relaxed.

21. A method of decreasing the pylorus lumen of the pyloric sphincter, comprising the steps of:
trans-pyloricly introducing an endoscope to a treatment site in the vicinity of the pyloric sphincter;
providing an access pathway through the mucosa; and
introducing a pyloric bulking agent into the wall of the pylorus below the mucosa that solidifies to form a non-biodegradable bulking implant, so that the pyloric bulking implant cooperates with the pyloric sphincter to substantially close the pylorus lumen to slow emptying of the stomach when the pyloric sphincter is relaxed.

22. A method for creating a restriction in the gastrointestinal tract extending from the pylorus through the colon in a body of a mammal to reduce food consumption, the gastrointestinal tract defined by a gastrointestinal tract wall having an interior mucosa, a submucosa and a muscle layer surrounding a tract lumen, the method comprising introducing at least one nonaqueous solution through the mucosa into the submucosa or muscle layer of the wall, and forming from the at least one nonaqueous solution a mass of non-biodegradable bulking agent within the gastrointestinal wall to reduce the cross-section of the tract lumen to slow passage of contents of the gastrointestinal tract.

23. The method of claim 22, wherein the at least one solution is a solution of a biocompatible polymer and a biocompatible solvent and wherein the forming step includes the step of precipitating the biocompatible polymer from the solution so that the biocompatible polymer solidifies in the tract wall to form a bulking agent mass and the biocompatible solvent disperses in the body.

24. The method of claim 22, for treating obesity wherein the introducing step includes the step of introducing the at least one nonaqueous solution into the tract wall in the vicinity of the pyloric sphincter.

25. The method of claim 24, wherein the forming step includes the step of forming a plurality of discrete non-biodegradable masses of bulking agent in the tract wall around the pylorus lumen.

26. The method of claim 24, wherein the at least one solution is a solution of a biocompatible polymer and a biocompatible solvent and wherein the forming step includes the step of precipitating the biocompatible polymer from the solution so that the biocompatible polymer solidifies in the tract wall and the biocompatible solvent disperses in the body.

27. The method of claim 22, further comprising a contrast agent in the solution for facilitating visualization of the non-biodegradable solid in the wall.

28. The method of claim 27, wherein the contrast agent is suspended in the solution.

29. The method of claim 22, wherein the forming step includes the step of forming a plurality of spaced-apart discrete non-biodegradable masses of bulking agent in the tract wall.

30. The method of claim 29, wherein the implants are spaced apart circumferentially around the tract wall.

31. A method for treating obesity by forming a restriction of the pylorus lumen in a body of a mammal to slow stomach emptying, the pylorus formed by a tract wall having a submucosal layer comprising the steps of introducing a solution of a biocompatible polymer and a biocompatible solvent into the submucosal layer and precipitating the biocompatible polymer from the solution so that the biocompatible polymer solidifies in the tract wall to form an implant in the tract wall that extends into and restricts the pylorus lumen.

32. The method of claim 31, wherein the implant is non-biodegradable.

33. The method of claim 31, wherein the precipitating step includes the step of forming a plurality of implants spaced circumferentially around the tract wall.

34. The method of claim 31, further comprising a contrast agent in the solution for facilitating visualization of the non-biodegradable solid in the wall.

35. The method of claim 34, wherein the contrast agent is suspended in the solution.

36. A method for creating a restriction in the gastrointestinal tract extending from the pylorus through the colon in a body of a mammal to reduce food consumption, the gastrointestinal tract defined by a gastrointestinal tract wall having an interior mucosa, a submucosa and a muscle layer surrounding a tract lumen, the method comprising introducing at least one nonaqueous solution through the mucosa into the submucosa or muscle layer of the wall in the vicinity of one of the pyloric sphincter, the first flexure of the duodenum, the duodenojunal flexure, and the ileocecal sphincter and forming from the at least one nonaqueous solution a mass of non-biodegradable bulking agent within the gastrointestinal wall to reduce the cross-section of the tract lumen to slow passage of contents of the gastrointestinal tract.

37 A method of treating obesity comprising providing a pyloric bulking device having a predetermined form and inserting the pyloric bulking device below the mucosa into the submucosa or muscle layer of the wall surrounding a lumen in the vicinity of one of the pyloric sphincter, the first flexure of the duodenum, the duodenojunal flexure, and the ileocecal sphincter to substantially close the lumen to slow emptying of the gastrointestinal tract.
